(19) United States
(12) Patent Application Publication
(10) Pub. No.: US 2002/0087027 A1
(43) Pub. Date: Jul. 4, 2002

(54) Esterification catalysts

(75) Inventors: Charles M. Lindall, Norton (GB); John Ridland, Durham (GB); Neville Slack, Billingham (GB)

Correspondence Address: Pillsbury Madison & Sutro LLP, Intellectual Property Group, East Tower, Ninth Floor, 1100 New York Avenue, N.W., Washington, DC, 20005-3918, US

(73) Assignee: ACMA Limited, Runcorn (GB)

(21) Appl. No.: 09/991,653

(22) Filed: Nov. 26, 2001

Related U.S. Application Data
(63) Continuation of application No. PCT/GB00/01674, filed on May 25, 2000.

(30) Foreign Application Priority Data
May 25, 1999 (GB) 9912210.3

(51) Int. Cl. C08F 4/42; B01J 31/12; C07C 69/52; C07C 69/02

(52) U.S. Cl. 560/205; 502/102; 502/171; 502/113; 502/114; 502/117; 560/231

(57) ABSTRACT

A catalyst composition suitable for use as a catalyst for the preparation of an ester comprises (a) an organometallic compound which is the reaction product of an orthoester or condensed orthoester of titanium, zirconium or aluminum, an alcohol containing at least two hydroxyl groups, an organophosphorus compound containing at least one P—OH group and preferably a base, and (b) a compound of germanium, antimony or tin. A process for the preparation of an ester comprises carrying out an esterification reaction in the presence of the catalyst composition. In a further embodiment the organometallic compound comprises the reaction product of, in addition, a 3-hydroxy carboxylic acid.

The invention concerns esterification catalyst compositions and in particular esterification catalyst compositions which comprise novel organotitanium, organozirconium or organoaluminium compounds in combination with other metal compounds.

Organotitanium compounds and, in particular, titanium alkoxides or orthoesters are known as catalysts for esterification processes. During the esterification, these compounds are frequently converted to insoluble compounds of titanium which result in a hazy product. The presence of a haze is a particular disadvantage in polyesters which have a high viscosity and/or high melting point and are therefore difficult to filter. Furthermore, many organotitanium compounds which are effective catalysts in the manufacture of polyesters such as polyethylene terephthalate are known to produce unacceptable yellowing in the final polymer. GBSA-2 314 081 relates to an esterification process in which these problems are partially solved but there is still a need for a catalyst system which induces little or no yellowing in a polyester produced using the catalyst.

It is an object of the present invention to provide an improved catalyst system for a process for preparing esters.

According to the invention, a catalyst composition suitable for use as a catalyst for the preparation of an ester comprises

(a) an organometallic compound which is the reaction product of an orthoester or condensed orthoester of at least one metal selected from titanium, zirconium or aluminium, an alcohol containing at least two hydroxyl groups, and an organophosphorus compound containing at least one P—OH group, and

(b) at least one compound of germanium, antimony or tin.

Also according to the invention, a process for the preparation of an ester comprises carrying out an esterification reaction in the presence of a catalyst composition comprising

(a) the reaction product of an orthoester or condensed orthoester of at least one metal selected from titanium, zirconium or aluminium, an alcohol containing at least two hydroxyl groups and an organophosphorus compound containing at least one P—OH group, and

(b) at least one compound of germanium, antimony or tin.

According to the invention, we also provide a polyester comprising the residues of a reaction between a polybasic acid or ester thereof with a polyhydric alcohol and further containing residues of a catalyst system comprising:

(a) the reaction product of an orthoester or condensed orthoester of at least one metal selected from titanium, zirconium or aluminium, an alcohol containing at least two hydroxyl groups and an organophosphorus compound containing at least one P—OH group, and

(b) at least one compound of germanium, antimony or tin.

In a further embodiment the organometallic compound suitable for use in an esterification process as component (a) of the aforementioned catalyst composition comprises the reaction product of an orthoester or condensed orthoester of at least one metal selected from titanium, zirconium or aluminium, an alcohol containing at least two hydroxyl groups, an organophosphorus compound containing at least one P—OH group and a 2-hydroxy carboxylic acid.

The organometallic compound suitable for use in an esterification process as component (a) of the aforementioned catalyst composition comprises the reaction product of an orthoester or condensed orthoester of at least one metal selected from titanium, zirconium or aluminium. Normally an orthoester or condensed orthoester of one of the selected metals is used but it is within the scope of the invention to use an orthoester or condensed orthoester of more than one of the selected metals. For clarity we refer hereinafter to a titanium, zirconium or aluminium orthoester or condensed orthoester, and all such references should be taken to include orthoesters or condensed orthoesters of more than one metal, e.g. to a mixture of titanium and zirconium orthoesters.

The organometallic compound which comprises component (a) of the catalyst composition of the invention is the reaction product of a titanium, zirconium or aluminium orthoester or condensed orthoester, an alcohol containing at least two hydroxyl groups, and an organophosphorus compound containing at least one P—OH group. Preferably, the orthoester has the formula M(OR)₄ or Al(OR)₃ where M is titanium or zirconium and R is an alkyl group. More preferably R contains 1 to 6 carbon atoms and particularly suitable orthoesters include tetraisopropoxy titanium, tetra-n-butoxy titanium, tetra-n-propoxy zirconium, tetra-n-butoxy zirconium and tri-isobutoxy aluminium.

The condensed orthoesters suitable for preparing the organometallic compounds used in this invention are typically prepared by careful hydrolysis of titanium, zirconium or aluminium orthoesters. Titanium or zirconium condensed orthoesters are frequently represented by the formula

R₁O[M(OR₁)₂O]nR₁

in which R¹ represents an alkyl group and M represents titanium or zirconium. Preferably, n is less than 20 and more preferably is less than 10. Preferably, R¹ contains 1 to 12 carbon atoms, more preferably, R¹ contains 1 to 6 carbon atoms and useful condensed orthoesters include the compounds known as polybutyl titanate, polyisopropyl titanate and polybutyl zirconate.

Preferably, the alcohol containing at least two hydroxyl groups is a dihydric alcohol and can be a 1,2-diol such as 1,2-ethanediol or 1,2-propanediol, a 1,3-diol such as 1,3-propanediol, a 1,4-diol such as 1,4-butanediol, a diol containing non-terminal hydroxyl groups such as 2-methyl-2,4-pentanediol or a dihydric alcohol containing a longer chain such as diethylene glycol or a polyethylene glycol. The preferred dihydric alcohol is 1,2-ethanediol. The organometallic compound can also be prepared from a polyhydric alcohol such as glycerol, trimethylolpropane or pentaerythritol.

Preferably, the organometallic compound which comprises component (a) of the catalyst composition is prepared by reacting a dihydric alcohol with an orthoester or condensed orthoester in a ratio of from 1 to 32 moles of dihydric alcohol to each mole of titanium, zirconium or aluminium. More preferably, the reaction product contains 2 to 25 moles of dihydric alcohol per mole of titanium, zirconium or aluminium (total) and most preferably 4 to 25 moles dihydric alcohol per mole of titanium, zirconium or aluminium (total).

The organophosphorus compound which contains at least one P—OH group can be selected from a number of organophosphorus compounds including phosphates, phosphate salts, pyrophosphates, phosphonates, phosphonate salts, phosphinates, phosphites and phosphorous derivatives of hydroxy carboxylic acids, eg. Citric acid.

Preferably, the organophosphorus compound is a salt of an alkyl or aryl phosphonate, a substituted or unsubstituted alkyl phosphate, a substituted or unsubstituted aryl phosphate or a phosphate of an alkylaryl glycol ether or an alkyl glycol ether or a substituted or unsubstituted mixed alkyl or aryl glycol phosphate. Useful compounds include tetrabutyl ammonium phenyl phosphonate, monoalkyl acid phosphates and dialkyl acid phosphates and mixtures of these. Convenient organophosphorus compounds are the compounds commercially available as alkyl acid phosphates and containing, principally, a mixture of mono- and di-alkyl phosphate esters. When an alkyl phosphate is used as the organophosphorus compound, the organic group preferably contains up to 20 carbon atoms, more preferably up to 8 carbon atoms and, most preferably, up to 6 carbon atoms. When alkylaryl or alkyl glycol ether phosphates are used the carbon chain length is preferably up to 18 carbon atoms and, more preferably, 6 to 12 carbon atoms.

Alternative organophosphorus compounds suitable for use in preparing the catalyst compositions of the invention are the reaction products obtainable by reacting phosphorus pentoxide and a polyhydric alcohol, particularly a glycol. Such products can be prepared by heating a mixture of phosphorus pentoxide and a polyhydric alcohol until a uniform liquid is formed. Conveniently, the amount of polyhydric alcohol used to prepare such a product is in excess of the stoichiometric amount required to fully react with the phosphorus pentoxide. The excess polyhydric alcohol acts as a solvent for the organophosphorus reaction product. Moreover, when a product containing excess polyhydric alcohol is used to prepare component (a) of the catalyst composition this excess polyhydric alcohol comprises at least a portion of the alcohol containing at least two hydroxyl groups used to prepare component (a). Suitable products contain up to 16 moles of polyhydric alcohol per mole of phosphorus (P). Preferably the products contain from 3 to 10 moles of polyhydric alcohol per mole of phosphorus.

Particularly preferred organophosphorus compounds include butyl acid phosphate, mixed butylethylene glycol phosphates, polyethylene glycol phosphate, aryl polyethylene glycol phosphates and a product of reaction of ethylene glycol and phosphorus pentoxide and the reaction product of an alkyl phosphonate and a hydroxy-functionalised carboxylic acid such as citric acid.

The amount of organophosphorus compound present in the reaction product which comprises component (a) of the catalyst composition of the invention is usually in the range 0.1 to 4.0 mole of phosphorus to 1 mole of metal (titanium, zirconium or aluminium), preferably in the range 0.1 to 2.0 mole phosphorus to 1 mole metal and most preferably in the range 0.1 to 1.0 mole phosphorus to 1 mole metal.

Preferably, the organometallic compound suitable for use in an esterification process as component (a) of the aforementioned catalyst composition additionally comprises a base, however when the organophosphorous compound comprises the reaction product of a base and a phosphate or phosphonate, it is not always essential to add a base to the components of the organometallic compound. For example, an alkali-metal salt or a quaternary ammonium salt of a phosphate or phosphonate may be used as the organophosphorus compound.

Suitable inorganic bases include metal hydroxides, e.g. sodium hydroxide, potassium hydroxide, calcium hydroxide, lithium hydroxide and ammonium hydroxide. Preferred organic bases include quaternary ammonium compounds such as tetrabutyl ammonium hydroxide, choline hydroxide (trimethyl(2-hydroxyethyl)ammonium hydroxide) or benzyltrimethyl ammonium hydroxide, or alkanolamines such as monoethanolamine, diethanolamine, triethanolamine and triisopropanolamine. Usually, the amount of base used is in the range 0.1 to 4.0 mole base per mole of metal (titanium, zirconium or aluminium). The preferred amount is in the range 0.1 to 2.0 mole base per mole of metal and frequently the amount of base present is in the range 0.1 to 1.0 mole base per mole of titanium, zirconium or aluminium.

When 2-hydroxy carboxylic acids are used to prepare the products which comprise component (a) of the catalyst of the invention, preferred acids used include lactic acid, citric acid, malic acid and tartaric acid. Some suitable acids are supplied as hydrates or as aqueous mixtures and can be used in this form. When a 2-hydroxy acid is present, the preferred molar ratio of acid to titanium, zirconium or aluminium in the reaction product is 0.5 to 4 moles per mole of titanium, zirconium or aluminium. More preferably the reaction product contains 1.0 to 3.5 moles of 2-hydroxy acid per mole of titanium, zirconium or aluminium.

The organometallic compound can be prepared by mixing the components (orthoester or condensed orthoester, alcohol containing at least two hydroxyl groups, organophosphorus compound and base, if present) with removal, if desired, of any by-product, (e.g. isopropyl alcohol when the orthoester is tetraisopropoxytitanium), at any appropriate stage. In one preferred method the orthoester or condensed orthoester and a dihydric alcohol are mixed and, subsequently, a base is added, followed by the organophosphorus compound. When a 2-hydroxy carboxylic acid is also present in the reaction product, this is usually added to the orthoester or condensed orthoester before the organophosphorus compound is added. Alternatively, all or part of the 2-hydroxy carboxylic acid can be neutralised with the base and the resulting salt added to the other components of the reaction mixture, including, if desired, a further portion of the base.

Component (b) of the catalyst composition of the invention is a compound of germanium, antimony or tin and, in general, any compound can be used including mixtures of compounds of more than one of these metals. The preferred compound of germanium is germanium dioxide. Preferably, the antimony compound is antimony trioxide or a salt of antimony, for example antimony triacetate. A number of tin compounds are suitable, including salts, such as tin acetate and organotin compounds, such as dialkyl tin oxides, for example, dibutyl tin oxide, dialkyl tin dialkanoates, for example, dibutyl tin dilaurate and alkylstannoic acids, for example butylstannoic acid (C₄H₉SnOOH).

A wide range of proportions of components (a) and (b) can be present in the catalyst composition of the invention. Generally, the molar ratio of component (a) to component (b) is in the range 9:1 to 1:9, and is preferably in the range 5:1 to 1:5, calculated as moles of Ti, Zr or Al to moles of Ge, Sb or Sn.

The esterification reaction of the process of the invention can be any reaction by which an ester is produced. The reaction may be (i) a direct esterification in which a carboxylic acid or its anhydride and an alcohol react to form an ester or (ii) a transesterification (alcoholysis) in which a first alcohol reacts with a first ester to produce an ester of the first alcohol and a second alcohol produced by cleavage of the first ester or (iii) a transesterification reaction in which two esters are reacted to form two different esters by exchange of alkoxy radicals. Direct esterification or transesterification can be used in the production of polymeric esters and a preferred process of the invention comprises a polyesterification process. Many carboxylic acids and anhydrides can be used in direct esterification including saturated and unsaturated monocarboxylic acids and anhydrides of such acids such as stearic acid, isostearic acid, capric acid, caproic acid, palmitic acid, oleic acid, palmitoleic acid, triacontanoic acid, benzoic acid, methyl benzoic acid, salicylic acid and rosin acids such as abietic acid, dicarboxylic acids such as phthalic acid, isophthalic acid, terephthalic acid, sebacic acid, adipic acid, azelaic acid, succinic acid, fumaric acid, maleic acid, naphthalene dicarboxylic acid and pamoic acid and anhydrides of these acids and polycarboxylic acids such as trimellitic acid, citric acid, trimesic acid, pyromellitic acid and anhydrides of these acids. Alcohols frequently used for direct esterification include aliphatic straight chain and branched monohydric alcohols such as butyl, pentyl, hexyl, octyl and stearyl alcohols, dihydric alcohols such as 1,2-ethanediol, 1,3-propanediol, 1,4-butanediol and 1,6 cyclohexane dimethanol and polyhydric alcohols such as glycerol and pentaerythritol.

The esters employed in an alcoholysis reaction are generally the lower homologues such as methyl, ethyl and propyl esters since, during the esterification reaction, it is usual to eliminate the displaced alcohol by distillation. These lower homologue esters of the acids suitable for direct esterification are suitable for use in the transesterification process according to the invention. Frequently (meth)acrylate esters of longer chain alcohols are produced by alcoholysis of esters such as methyl acrylate, methyl methacrylate, ethyl acrylate and ethyl methacrylate- Typical alcohols used in alcoholysis reactions include butyl, hexyl, n-octyl and 2-ethyl hexyl alcohols and substituted alcohols such as dimethylaminoethanol.

When the esterification reaction is a transesterification between two esters, generally the esters will be selected so as to produce a volatile product ester which can be removed by distillation.

As mentioned hereinbefore, polymeric esters can be produced by processes involving direct esterification or transesterification and a particularly preferred embodiment of the esterification process of the invention is a polyesterification reaction in the presence of the catalyst composition described hereinbefore. In a polyesterification reaction polybasic acids or esters of polybasic acids are usually reacted with polyhydric alcohols to produce a polymeric ester. Linear polyesters are often produced from dibasic acids such as those mentioned hereinbefore or esters of said dibasic acids and dihydric alcohols. Preferred polyesterification reactions according to the invention include the reaction of terephthalic acid or dimethyl terephthalate with 1,2-ethanediol (ethylene glycol) to produce polyethylene terephthalate or with 1,3-propanediol (propylene glycol) to produce polypropylene terephthalate or with 1,4-butanediol (butylene glycol) to produce polybutylene terephthalate or reaction of naphthalene dicarboxylic acid or dimethyl naphthalenate with 1,2-ethanediol to produce polyethylene naphthalenate. Other acids, such as isophthalic acid and other glycols such as 1,6 cyclohexane dimethanol and polyhydric alcohols such as glycerol, trimethylolpropane and pentaerythritol are also suitable for preparing polyesters.

The catalyst composition of the invention comprises two components, (a) and (b) and these may be premixed to form the catalyst composition of this invention before the composition is mixed with the reactants for an esterification reaction. Alternatively, components (a) and (b) can be separately added to the reactants in order to carry out an esterification reaction according to this invention.

The esterification reaction of the invention can be carried out using any appropriate, known technique for an esterification reaction.

A typical process for the preparation of polyethylene terephthalate comprises two stages. In the first stage terephthalic acid or dimethyl terephthalate is reacted with 1,2-ethanediol to form a prepolymer and the by-product water or methanol is removed. The prepolymer is subsequently heated in a second stage to remove 1,2-ethanediol and form a long chain polymer. Either or both these stages may comprise an esterification process according to this invention.

In direct esterificatlon the acid or anhydride and an excess of alcohol are typically heated, if necessary in a solvent, in the presence of the catalyst composition. Water is a by-product of the reaction and this is removed, as an azeotrope with a boiling mixture of solvent and/or alcohol. Generally, the solvent and/or alcohol mixture which is condensed is at least partially immiscible with water which is therefore separated before solvent and/or alcohol are returned to the reaction vessel. When reaction is complete the excess alcohol and, when used, solvent are evaporated. In view of the fact that the catalyst compositions of the invention do not normally form insoluble species, it is not generally necessary to remove them from the reaction mixture, as is frequently necessary with conventional catalysts. A typical direct esterification reaction is the preparation of bis(2-ethylhexyl) phthatate which is prepared by mixing phthalic anhydride and 2-ethyl hexanol. An initial reaction to form a monoester is fast, but the subsequent conversion of the monoester to diester is carried out by refluxing in the presence of the catalyst composition at a temperature of 180-200° C. until all the water has been removed. Subsequently the excess alcohol is removed.

In an alcoholysis reaction, the ester, first alcohol and catalyst composition are mixed and, generally, the product alcohol (second alcohol) is removed by distillation, often as an azeotrope with the ester. Frequently it is necessary to fractionate the vapour mixture produced from the alcoholysis in order to ensure that the second alcohol is separated effectively without significant loss of product ester or first alcohol. The conditions under which alcoholysis reactions are carried out depend principally upon the components of the reaction and generally components are heated to the boiling point of the mixture used.

A preferred process of the invention is the preparation of polyethylene terephthalate. A typical batch production of polyethylene terephthalate is carried out by charging terephthalic acid and ethylene glycol to a reactor along with catalyst composition, if desired, and heating the contents to 260-270° C. under a pressure of about 0.3 MPa. Reaction commences as the acid dissolves at about 230° C. and water is removed. The product is transferred to a second autoclave reactor and catalyst composition is added, if needed. The reactor is heated to 285-310° C. under an eventual vacuum of 100 Pa to remove ethylene glycol by-product. The molten product ester is discharged from the reactor, cooled and chipped. The chipped polyester may be then subjected to solid state polymerisation, if appropriate.

A preferred means of adding the catalyst compositions of this invention to a polyesterification reaction is in the form of a slurry in the glycol being used (e.g. ethylene glycol in the preparation of polyethylene terephthalate). Components (a) and (b) can be added to the reaction mixture as separate slurries or mixed to prepare a slurry containing both components, which slurry is then added to the reactants. This method of addition is applicable to addition of the catalyst composition to the polyesterification reaction at the first stage or at the second stage.

The amount of catalyst used in the esterification process of the invention generally depends upon the total metal content (expressed as amount of Ti, Zr or Al plus amount of Ge, Sb or Sn) of the catalyst composition. Usually the amount is from 10 to 1200 parts per million (ppm) of metal based on weight of product ester for direct or transesterification reactions. Preferably, the amount is from 10 to 650 ppm of total metal based on weight of product ester. In polyesterification reactions the amount used is generally expressed as a proportion of the weight of product polyester and is usually from 5 to 550 ppm expressed as total metal (Ti, Zr or Al plus Ge, Sb or Sn) based on product polyester. Preferably, the amount is from 5 to 300 ppm expressed as total metal based on product polyester.

Generally, the amount of Ti, Zr or Al used in a direct esterification or transesterification will be in the range 5 to 500 ppm Ti, Zr or Al and more preferably in the range 5 to 250 ppm Ti, Zr or Al, based on product ester; and the amount of Ge, Sb or Sn used in a direct esterification or transesterification will be in the range 5 to 700 ppm Ge, Sb or Sn, preferably in the range 5 to 400 ppm Ge, Sb or Sn, based on product ester. For polyesterification, the preferred amount of Ti, Zr or Al is in the range 3 to 250 ppm Ti, Zr or Al based on product polyester and, more preferably, the amount is 3 to 100 ppm Ti. Zr or Al based on product polyester. The preferred amount of Ge, Sb or Sn used in polyesterification is in the range 3 to 300 ppm Ge, Sb or Sn and more preferably is in the range 5 to 200 ppm Ge, Sb or Sn based on product polyester.

The products of this invention have been shown to be effective catalyst compositions for producing esters and polyesters at an economical rate without leading to haze in the final product and with a reduced amount of yellowing of polyesters in comparison to known catalysts.

The invention is illustrated by the following examples.

### Preparation of Organometallic Compounds for use in Catalyst Compositions

Example 1

Ethylene glycol (49.6 g, 0.8 moles) was added from a dropping funnel to stirred titanium n-butoxide (34 g, 0.1 mole) in a 250 ml flask fitted with stirrer, condenser and thermometer. An aqueous solution of sodium hydroxide, containing 32% NaOH by weight, (12.5 g, 0.1 mole) was added to the reaction flask slowly with mixing to yield a clear yellow liquid. To this liquid was added a butyl/ethylene glycol mixed phosphoric acid mono/diester with a low phosphorus content available under the trade name HORDAPHOS DGB[LP] from Clariant AG, (11.82 g, 0.05 mole of phosphorus). Ti content of 4.43% by weight.

Example 2

Ethylene glycol (100 g, 1.6 moles) was added from a dropping funnel to stirred titanium n-butoxide (34 g, 0.1 mole) in a 250 ml conical flask fitted with stirrer. An aqueous solution of sodium hydroxide, containing 32% NaOH by weight (12.5 g, 0.1 mole) was added drop-wise to the reaction flask with mixing to yield a clear pale yellow liquid. To this liquid a combined reaction product of P₂O₅ (7.1 g, 0.05 mole) and ethylene glycol (55 g, 0.9 moles) was slowly added and the resulting mixture was stirred for several minutes. The P₂O₅ reaction product was prepared by dissolving P₂O₅ in ethylene glycol, with a combination of mixing and carefully controlled heating; this was subsequently allowed to cool. After removing n-butanol at 70° C. under vacuum to constant weight the product was a pale yellow liquid with a Ti content of 2.96% by weight.

Example 3

Ethylene glycol (49.6 g, 0.8 moles) was added from a dropping funnel to stirred titanium n-butoxide (34 g, 0.1 mole) in a 250 ml conical flask fitted with stirrer. An aqueous solution of sodium hydroxide, containing 32% NaOH by weight (12.5 g, 0.1 mole) was added drop-wise to the reaction flask with mixing to yield a clear pale yellow liquid. To this liquid a combined reaction product of P₂O₅ (3.55 g, 0.025 mole) and ethylene glycol (49.6 g, 0.8 mole) was slowly added and the resulting mixture was stirred for several minutes. The P₂O₅ reaction product was prepared by dissolving the P₂O₅ in ethylene glycol, with a combination of mixing and carefully controlled heating; this was subsequently allowed to cool. After removing n-butanol at 70° C. under vacuum to constant weight the product was a pale yellow liquid with a Ti content of 4.49% by weight.

Example 4

Ethylene glycol (99.2 g, 1.6 moles) was added from a dropping funnel to stirred titanium n-butoxide (68 g, 0.2 moles) in a 250 ml flask fitted with stirrer, condenser and thermometer. An aqueous solution of sodium hydroxide, containing 32% NaOH by weight, (25 g, 0.2 mole) was added to the reaction flask slowly with mixing to yield a clear yellow liquid. To this liquid was added an aryl polyethylene glycol phosphate available commercially under the trade name HORDAPHOS P123 from Clariant AG, (86.32 g, 0.128 moles of phosphorus) and the resulting mixture was stirred for several minutes to produce a pale yellow liquid with a Ti content of 3.44% by weight.

Example 5

Ethylene glycol (496.0 g, 8.00 moles) was added from a dropping funnel to stirred titanium n-butoxide (340 g, 1.00 mole) in a 1 liter fishbowl flask fitted with stirrer, condenser and thermometer. An aqueous solution of sodium hydroxide, containing 32% NaOH by weight, (125 g, 1.00 mole) was added to the reaction flask slowly with mixing to yield a clear pale yellow liquid. To this liquid was then added a butyl acid phosphate, (91.0 g, 0.50 mole of phosphorus) and the resulting mixture was stirred for 1 hour to produce a pale yellow liquid with a Ti content of 4.56% by weight.

Example 6

Ethylene glycol (49.6 g, 0.8 moles) was added from a dropping funnel to stirred titanium n-butoxide (4 g, 0.1 moles) in a 250 ml flask fitted with stirrer, condenser and thermometer. Choline hydroxide (26.93 g, 0.1 mole) was added to the reaction flask slowly with mixing to yield a clear yellow liquid. To this liquid was added a di-butyl acid phosphate having a carbon length of 4 carbon atoms, (10.5 g, 0.05 moles of phosphorus) and the resulting mixture was stirred for several minutes to produce a pale yellow liquid with a Ti content of 3.96% by weight.

Example 7

Citric acid (38.3 g, 0.2 mol) was dissolved in the hot water (22 g, 1.22 mol). TIPT (28.4 g, 0.1 mol) was added slowly over 10 minutes. BAYHIBlT™ AM (available from Bayer), which is 2-phosphonobutane-1,2,3-tricarboxylic acid (a 49% solution in water) (27.6 g, 0.05 mol, including 0.78 mol water) was added slowly over 10 minutes to give a white suspension. The mixture was refluxed at about 85° C. for 60 minutes to give a clear pale yellow solution. Water/IPA was distilled off at atmospheric pressure until a head temperature of ˜95° C. was attained. The solution was allowed to cool to ˜60° C., before a 32% sodium hydroxide solution (37.5 g, 0.3 mol) was slowly added over 10 minutes. Ethylene glycol (50 g, 0.8 mol) was then added and the remaining water/IPA removed by heating under vacuum. The final product was a clear pale yellow liquid. Some precipitated solids were observed after 48 hours. These solids were redissolved by adding a further 8 equivalents of MEG to yield a clear liquid with a Ti content of 2.91% by weight.

### Polyesterification

Example 8

A polycondensation reaction was carried out in a mechanically-stirred 300 ml glass vessel fitted with side arm and cold trap for collection of monoethyleneglycol. A thermostatically controlled ceramic heating element was used to provide heat and an oil vacuum pump was connected to the cold trap. A nitrogen blanket was provided via a connection to the cold trap.

Polyethylene terephthalate was prepared from pure bis(hydroxyethyl)- terephthalate polymer precursor.

100 g of bis(hydroxyethyl)terephthalate polymer precursor was placed in the reaction flask under a nitrogen flow, followed by a dilute solution of catalyst component (Ti added at 15 ppm, Ge at 50 ppm, Sb at 125 ppm and Sn at 15 ppm for mixed catalysts) in monoethyleneglycol. For the unmixed catalysts (Table 2) the levels of the single metals were doubled (ie Ti added at 30 ppm, Ge at 100 ppm, Sb at 250 ppm and Sn at 30 ppm). This was heated with stirring to 250° C. for 20-25 minutes at which point a stabiliser (phosphoric acid, calculated to produce the equivalent of 32 ppm P in the mixture, making allowance for P content of catalyst composition) again as a solution in monoethyleneglycol. The nitrogen flow was stopped and vacuum applied steadily to 100 Pa. After 20-25 minutes the temperature was increased steadily from 250° C. to 290° C. As the reaction progressed the current required to maintain a constant stirrer speed increased up to a value of 109 mA, at which point reaction was deemed to be complete. The vacuum was then broken with nitrogen and the molten polymer discharged and quenched into cold water. It was then dried for 12 hours at 65° C.

### Polymer Analysis

The colour of the polymer was measured using a Byk-Gardner Colourview spectrophotometer. A common model to use for colour expression is the Cielab L*, a* and b* scale where the b-value describes yellowness. The yellowness of the polymer increases with b-value.

The polymer intrinsic viscosities were measured by glass capillary viscometry using 60/40 phenol/1,1,2,2-tetrachlorethane as solvent. The polymers were examined by ¹H NMR spectroscopy to determine the amount of diethylene glycol (DEG) residues present in the polymer chain (expressed as weight percent of polymer), the proportion of hydroxyl (OH) end groups present (expressed as number of end groups per 100 polymer repeating units) and the proportion of vinyl end groups (VEG) present (expressed as number of end groups per 100 polymer repeating units). The results are shown in Tables 1 & 2.

TABLE 1
Example 8 Polyesterification-Mixed catalysts

| | Reaction | | | | Intrinsic | | | |
|---|---|---|---|---|---|---|---|---|
| Catalyst | Time | Colour | | | Viscosity | NMR | | |
| Composition | (Minutes) | L* | a* | b* | dl/g | DEG | OH | VEG |
| Example 1 + GeO₂ | 140 | 55.34 | −0.69 | 5.86 | 0.36 | 2.45 | 2.01 | 0.020 |
| Example 2 + GeO₂ | 156 | 63.42 | −0.77 | 2.84 | 0.40 | 2.43 | 2.59 | 0.003 |
| Example 3 + GeO₂ | 127 | 56.29 | −0.61 | 3.49 | 0.40 | 2.30 | 2.63 | 0.003 |
| Example 4 + GeO₂ | 230 | 70.06 | −0.81 | 12.28 | 0.39 | 2.67 | 2.73 | 0.021 |
| Example 1 + | 148 | 65.39 | −0.76 | 11.45 | 0.35 | 2.45 | 2.86 | 0.004 |
| antimony acetate | | | | | | | | |
| Example 2 + | 160 | 61.02 | −0.02 | 5.48 | 0.43 | 2.40 | 2.30 | 0.003 |
| antimony acetate | | | | | | | | |
| Example 3 + | 170 | 63.64 | −1.17 | 5.15 | 0.44 | 2.64 | 2.05 | 0.010 |
| antimony acetate | | | | | | | | |
| Example 3 + dibutyl | 160 | 63.13 | −1.14 | 4.58 | | 2.37 | 1.82 | ND |
| tin oxide | | | | | | | | |
| Example 1 + dibutyl | 160 | 65.89 | −1.07 | 10.79 | | 2.35 | 2.28 | 0.009 |
| tin oxide | | | | | | | | |
| Example 2 + dibutyl | 160 | 65.57 | −1.22 | 8.50 | | 2.67 | 2.73 | 0.003 |
| tin oxide | | | | | | | | |

ND = Not detected

TABLE 2
Example 8 Comparative Examples: Polyesterication-Pure Catalysts

| | Reaction | | | | Intrinsic | | | |
|---|---|---|---|---|---|---|---|---|
| Catalyst | Time | Colour | | | Viscosity | NMR | | |
| Composition | (Minutes) | L* | a* | b* | dl/g | DEG | OH | VEG |
| Example 1 | 130 | 56.3 | −0.9 | 5.2 | 0.39 | 2.55 | 2.62 | 0 |
| (30 ppm Ti) | | | | | | | | |
| Example 2 | 160 | 58.9 | −0.9 | 6.4 | 0.4 | 2.6 | 2.8 | 0 |
| (30 ppm Ti) | | | | | | | | |
| Example 3 | 135 | 55.6 | −0.8 | 5.2 | 0.42 | 2.43 | 2.04 | <0.003 |
| (30 ppm Ti) | | | | | | | | |
| Example 4 | 130 | 67.7 | −0.8 | 6.4 | 0.43 | 2.46 | 2.44 | 0 |
| (30 ppm Ti) | | | | | | | | |
| Example 6 | 135 | 62.62 | −0.92 | 10.24 | 0.45 | 2.41 | 1.69 | 0.0140 |
| (30 ppm Ti) | | | | | | | | |
| antimony acetate | 170 | 50.1 | −0.9 | 3.7 | 0.4 | 2.7 | 2.8 | 0 |
| (250 ppm Sb) | | | | | | | | |
| germanium oxide | >250 | 58.9 | −1 | 7.7 | — | — | — | — |
| (100 ppm Ge) | | | | | | | | |
| dibutyl tin oxide | >250 | 60.1 | −7.5 | 3.2 | 0.3 | 2.62 | 2.64 | <0.003 |
| (30 ppm Sn) | | | | | | | | |

Example 9

The catalysts were used to prepare polyethylene terephthalate (PET). Ethylene glycol (2.04 kg) and terephthalic acid (4.55 kg) were charged to a stirred, jacketed reactor. The catalyst and other additives, including a DEG suppressant, were added and the reactor heated to 226-252° C. at a pressure of 40 psi to initiate the first stage direct esterification (DE) process. Water was removed as it was formed with recirculation of the ethylene glycol. On completion of the DE reaction the contents of the reactor were allowed to reach atmospheric pressure before a vacuum was steadily applied. The stabilisers were added and the mixture heated to 290±2° C. under vacuum to remove ethylene glycol and yield polyethylene terephthalate. The final polyester was discharged through a lace die, water cooled and chipped once a constant torque which indicated an IV of around 0.62 had been reached. Samples of polymer were collected at 5, 20 and 30 minutes from commencing discharge to monitor polymer stability during the process of casting from the reactor. Colour values were measured for each sample and are shown in Table 4.

Colour, IV and NMR data of polyesters made in Example 9 are given in Tables 3 & 4. Heat-cool differential scanning calorimetry (DSC) experiments on ‘re-quenched’ samples were conducted as follows: 10 mg samples were dried at 80° C. in a vacuum oven. These dried samples were then held at 290° C. for 2 minutes in a Perkin-Elmer DSC instrument, before being quenched onto the cold block (−40° C.). The re-quenched samples were then subjected to a heat/hold 2 minutes/cool procedure, at heating & cooling rates of 20° C./minute on a Perkin-Elmer DSC 7a. The cooling data quoted have been corrected by adding 2.8° C. to the computer-generated temperatures. Molecular weights were determined by gel permeation chromatography (GPC). The DSC results for all catalysts tested in the reaction described in Example 9 are presented in Table 5.

Examining Tables 1-4 it is evident that combining the titanium - phosphorous catalysts with the other metal catalysts gives polyester of lower yellowness (b value) than expected. There is a benefit to be obtained in reducing the amount of antimony used in polyesters which are used for applications in which the perceived potential for antimony to migrate from the material may cause problems. Also the high cost of germanium catalysts make it desirable to reduce the amount of germanium used in polyester catalysis. We have demonstrated that lower levels of these materials may be used without loss of effectiveness by replacing at least a part thereof with titanium, zirconium or aluminium catalysts without the unacceptable rise in polymer yellowness which might normally be expected from using increased amounts of these materials, particularly titanium.

TABLE 5
Example 8 Polyesterification DSC Results

| | | | | Heat | | | | | | Cool | | |
|---|---|---|---|---|---|---|---|---|---|---|---|---|
| | | Other | | Tgₒ | Tnₒ | Tn | ΔH | Tp | ΔH | Tc | Tcₒ | ΔH |
| Example | ppm Ti | Catalyst | ppm M | ° C. | ° C. | ° C. | J/g | ° C. | J/g | ° C. | ° C. | J/g |
| Ex 2 | 30 | GeO₂ | 50 | 77 | 141 | 152 | −38 | 254 | 41 | 165 | 198 | −24 |
| Ex 2 | 30 | Sb(OAc)₃ | 150 | 77 | 137 | 151 | −40 | 253 | 41 | 186 | 207 | −48 |
| Ex 2 | 15 | GeO₂ | 50 | 77 | 146 | 158 | −38 | 252 | 40 | 165 | 194 | −24 |
| Ex 2 | 15 | Sb(OAc)₃ | 150 | 76 | 143 | 154 | −38 | 253 | 41 | 186 | 206 | −47 |
| Ex 5 | 15 | GeO₂ | 50 | 76 | 141 | 153 | −38 | 253 | 41 | 165 | 195 | −29 |
| Ex 5 | 30 | GeO₂ | 50 | 76 | 140 | 151 | −38 | 252 | 40 | 166 | 196 | 32 |
| Ex 5 | 15 | Sb(OAc)₃ | 150 | 76 | 141 | 152 | −38 | 253 | 41 | 186 | 206 | −44 |
| Ex 2 | 30 | — | — | 73 | 141 | 154 | −38 | 246 | 38 | 164 | 192 | −34 |
| | 0 | Sb(OAc)₃ | 350 | 77 | 144 | 156 | −38 | 253 | 40 | 183 | 203 | −43 |

Tg₀ = polymer glass transition temperature, Tnₒ = onset of crystallisation (heating), Tn = crystallisation peak (heating), Tg = melting point, Tcₒ = onset of crystallisation (cooling), Tc = crystallisation (cooling), ΔH = enthalpy change, Tₚ = peak (melting) temperature.

Examining Table 5 it is evident that the crystallisation temperatures for polyesters made with a mixed antimony/titanium catalyst are always high during cooling and always low during heating cycles, when compared with polyesters produced using the titanium catalyst and mixed titanium/germanium catalysts. This is known in the art and is because the higher levels of antimony used may give rise to high levels of catalyst residues which act as nucleating points for crystallisation. Titanium and germanium are known as more soluble catalysts and are used at lower levels. Lower residues are therefore present causing less facile crystallisation. A surprising feature of this invention is that the crystallisation temperatures for polyesters made with a mixed antimony/titanium catalyst are always high during cooling and always low during heating, when compared with polyesters produced using only antimony acetate as catalyst. The levels of antimony used in the antimony acetate catalyst are double the level of the combined antimony and titanium in the mixed catalyst and would therefore be expected to cause more facile crystallisation. It is therefore likely that either a synergistic effect between the titanium and antimony or a distinct change in the polymer architecture causes more facile crystallisation. Control over the rate of crystallisation in polyesters may result in higher polyester throughput during several processing applications.

TABLE
Example 9 Polysterification Polymer properties

| | | | | | | | | | | OH | Vinyl | | | | | | |
|---|---|---|---|---|---|---|---|---|---|---|---|---|---|---|---|---|---|
| | | Other metal | | ppm P | | | | | | ends | Ends | | | | | | |
| TI | ppm | (M) | ppm | | As | DE | PC | | wt % | /100 | /100 | Mn | Mw | Mw/ | Tg | Tc | Tm |
| Catalyst | Ti | Compound | M | In Catalyst | H₃PO₄ | Time | Time | I.V. | DEG | units | units | 1000's | 1000's | Mn | ° C. | ° C. | ° C. |
| Ex. 2 | 30 | GeO₂ | 50 | 19 | — | 75 | 114 | 0.6 | 0.94 | 1.3 | 0.039 | 23.7 | 63.5 | 2.68 | 77 | 165 | 254 |
| Ex. 2 | 30 | Sb(OAc)₃ | 150 | 19 | — | 99 | 120 | 0.62 | 1.08 | 1.46 | 0.017 | 44.1 | 71.7 | 1.63 | 77 | 186 | 253 |
| Ex. 2 | 15 | GeO₂ | 50 | 9.5 | — | 86 | 143 | 0.6 | 1.01 | 1.16 | 0.041 | 28.5 | 66.7 | 2.34 | 77 | 165 | 252 |
| Ex. 2 | 15 | Sb(OAc)₃ | 150 | 9.5 | — | 86 | 125 | 0.6 | 1.09 | 1.41 | 0.016 | 28.3 | 68.2 | 2.41 | 76 | 186 | 253 |
| Ex. 5 | 15 | GeO₂ | 50 | 5 | 3 | 84 | 108 | 0.61 | 1.04 | 1.27 | 0.007 | 28.6 | 64.8 | 2.27 | 76 | 165 | 253 |
| Ex. 5 | 30 | GeO₂ | 50 | 10 | 6 | 88 | 110 | 0.62 | 1.36 | 1.31 | 0.006 | 27.2 | 63.7 | 2.34 | 76 | 166 | 252 |
| Ex. 5 | 15 | Sb(OAc)₃ | 150 | 5 | 3 | 119 | 111 | 0.61 | 1.08 | 1.38 | 0.010 | 23.1 | 64.5 | 2.79 | 76 | 186 | 253 |
| Ex. 5 | 30 | Sb(OAc)₃ | 150 | 10 | 6 | 85 | 77 | 0.62 | 1.11 | 1.37 | 0.009 | 31.3 | 63.5 | 2.03 | 77 | 179 | 253 |
| Ex. 2 | 30 | — | — | 19 | 0 | 92 | 152 | 0.62 | 2.62 | 1.18 | 0.040 | 30.1 | 78.4 | 2.60 | 73 | 164 | 246 |
| 0 | 0 | Sb(OAc)₃ | 350 | 0 | 10 | 100 | 129 | 0.62 | 1.22 | 1.28 | 0.010 | 28.4 | 74.9 | 2.64 | 77 | 183 | 253 |
| Ex. 5 | 30 | — | — | 10 | 6 | 98 | 92 | 0.6 | 2.08 | 1.26 | 0.022 | — | — | — | — | — | — |

TABLE 4
Example 9 Polyesterification-Polymer Colour properties

| | | | | ppm P | | | | | | | | | | |
|---|---|---|---|---|---|---|---|---|---|---|---|---|---|---|
| Ti | | Other | | in | | 5 minutes | | | 20 minutes | | | 30 minutes | | |
| Catalyst | ppm Ti | Cat | ppm M | catalyst | (H₃PO₄) | L | a | b | L | a | b | L | a | b |
| Ex. 2 | 30 | GeO₂ | 50 | 19 | — | 67.44 | −1.21 | 6.52 | 66.87 | −1.25 | 12.83 | 65.52 | −0.43 | 16.98 |
| Ex. 2 | 30 | Sb(OAc)₃ | 150 | 19 | — | 67.00 | −2.10 | 11.44 | 65.89 | −1.38 | 17.41 | 58.31 | 2.06 | 23.59 |
| Ex. 2 | 15 | GeO₂ | 50 | 9.5 | — | 67.43 | −1.80 | 7.96 | 68.45 | −2.02 | 11.43 | 67.39 | −1.53 | 15.25 |
| Ex. 2 | 15 | Sb(OAc)₃ | 150 | 9.5 | — | 63.41 | −2.87 | 10.63 | 65.27 | −2.79 | 11.65 | 62.62 | −2.53 | 14.96 |
| Ex. 5 | 15 | GeO₂ | 50 | 5 | 3 | 68.20 | −2.19 | 8.35 | 67.14 | −2.17 | 15.00 | 64.66 | −0.94 | 19.13 |
| Ex. 5 | 30 | GeO₂ | 50 | 10 | 6 | 69.74 | −2.78 | 14.54 | 66.54 | −1.36 | 20.85 | 63.81 | 0.17 | 24.38 |
| Ex. 5 | 15 | Sb(OAc)₃ | 150 | 5 | 3 | 66.13 | −3.14 | 12.24 | 64.26 | −2.52 | 16.84 | 61.21 | −0.62 | 22.93 |
| Ex. 5 | 30 | Sb(OAc)₃ | 150 | 10 | 6 | 61.92 | −2.74 | 20.49 | 61.17 | −1.49 | 24.32 | 57.18 | 0.99 | 27.33 |
| Ex. 2 | 30 | — | — | 19 | 0 | 67.86 | −2.57 | 14.23 | 75.85 | −2.44 | 12.44 | 73.83 | −2.44 | 14.98 |
| 0 | 0 | Sb(OAc)₃ | 350 | 0 | 10 | 69.06 | −1.90 | 4.69 | 65.61 | −2.38 | 6.26 | 67.64 | −2.49 | 7.76 |
| Ex. 5 | 30 | — | — | 10 | 6 | 67.65 | −2.18 | 11.21 | 67.53 | −2.05 | 14.64 | 66.80 | −2.09 | 16.88 |

What is claimed is:

1 A catalyst composition suitable for use as a catalyst for the preparation of an ester comprising
(a) an organometallic compound which is the reaction product of an orthoester or condensed orthoester of at least one metal selected from titanium, zirconium or aluminium, an alcohol containing at least two hydroxyl groups, and an organophosphorus compound containing at least one p-oh group, and
(b) at least one compound of germanium, antimony or tin.

2 A catalyst composition according to claim 1 characterized in that the organometallic compound comprises the reaction product of an orthoester or condensed orthoester of at least one metal selected from titanium, zirconium or aluminium, an alcohol containing at least two hydroxyl groups, an organophosphorus compound containing at least one P—OH group, and a base.

3 A catalyst composition according to claim 1 or claim 2 characterized in that the organometallic compound comprises the reaction product of an orthoester or condensed orthoester of at least one metal selected from titanium, zirconium or aluminium, an alcohol containing at least two hydroxyl groups, an organophosphorus compound containing at least one P—OH group, a base and a 2-hydroxy carboxylic acid.

4 A catalyst composition according to claim 3 characterized in that the 2-hydroxy acid is lactic acid, citric acid, malic acid or tartaric acid or a phosphorus derivative of at least one of said acids.

5 A catalyst composition according to claim 1, characterized in that the orthoester has the formula M(OR)₄ and/or Al(OR)₃ where M is titanium and/or zirconium and R is an alkyl group containing from 1 to 6 carbon atoms.

6 A catalyst composition according claim 1 characterized in that the condensed orthoester has a structure which can be represented by the formula, R¹O[M(OR¹)₂O]ₙR¹ where M is titanium and/or zirconium, R¹ is an alkyl group containing 1 to 6 carbon atoms and n is less than 20.

7 A catalyst composition according to claim 1, characterized in that the alcohol containing at least two hydroxyl groups is 1,2-ethanediol, 1,2-propanediol, 1,3-propanediol, 1,4-butanediol, 2-methyl-2,4-pentanediol, diethylene glycol, polyethylene glycol, glycerol, trimethylolpropane, pentaerythritol or 1,6 cyclohexane dimethanol.

8. A catalyst composition according to claim 1, characterized in that the organometallic compound is prepared by reacting a dihydric alcohol with an orthoester or condensed orthoester in a ratio of from 1 to 32 moles of dihydric alcohol to each mole of titanium, zirconium or aluminium.

9. A catalyst composition according to claim 1, characterized in that the organophosphorus compound is a phosphate, a pyrophosphate, a phosphonate, a phosphinate, a phosphite or a salt of a phosphate or phosphonate or a phosphorous derivative of a hydroxy acid.

10. A catalyst composition according to claim 9, characterized in that the organophosphorus compound is a substituted or unsubstituted alkyl phosphate, a substituted or unsubstituted aryl phosphate, a salt of an alkyl or aryl phosphonate, a phosphate of an alkylaryl glycol ether or an alkyl glycol ether, or a product obtainable by reaction of phosphorus pentoxide with a polyhydric alcohol.

11. A catalyst composition according to claim 10, characterized in that the organophosphorus compound is an alkyl phosphate in which the organic group contains up to 20 carbon atoms.

12. A catalyst composition according to claim 10, characterized in that the organophosphorus compound is a phosphate of an alkylaryl glycol ether or an alkyl glycol ether having a carbon chain length up to 18 carbon atoms.

13. A catalyst composition according to claim 10, characterized in that the organophosphorus compound is a reaction product of phosphorus pentoxide and a polyhydric alcohol in which the molar ratio of polyhydric alcohol to P is up to 50:1.

14. A catalyst composition according to claim 9, characterized in that the organophosphorus compound is a phosphorous derivative of a hydroxy acid

15. A catalyst composition according to claim 1, characterized in that the organophosphorus compound is present in the organometallic compound in an amount in the range 0.1 to 4.0 mole of phosphorus to 1 mole of titanium, zirconium or aluminium.

16. A catalyst composition according to claim 1, characterized in that a base is present in the organometallic compound in an amount in the range 0.01 to 4.0 mole of base to 1 mole of titanium, zirconium or aluminium.

17. A catalyst composition according to claim 3, characterized in that the 2-hydroxy acid is present in the organometallic compound in an amount in the range 0.1 to 4 mole acid to 1 mole of titanium, zirconium or aluminium.

18. A catalyst composition according to claim 1, characterized in that the compound of germanium is germanium dioxide or a salt of germanium.

19. A catalyst composition according to claim 1, characterized in that the compound of antimony is antimony trioxide or a salt of antimony.

20. A catalyst composition according to claim 1, characterized in that the compound of tin is a tin salt, a dialkyl tin oxide, a dialkyl tin dialkanoate or an alkylstannoic acid.

21. A catalyst composition according to claim 1, characterized in that the molar ratio of the organometallic compound to the compound of germanium, antimony or tin is in the range 9:1 to 1:9 calculated as moles of Ti, Zr or Al to moles of Ge, Sb or Sn.

22. A process for the preparation of an ester comprising carrying out an esterification reaction in the presence of a catalyst composition comprising
(a) the reaction product of an orthoester or condensed orthoester of at least one metal selected from titanium, zirconium or aluminium, an alcohol containing at least two hydroxyl groups, an organophosphorus compound containing at least one P—OH group and optionally a base, and
(b) at least one compound of germanium, antimony or tin.

23. A process according to claim 22 characterized in that the esterification reaction comprises reaction of an alcohol with stearic acid, isostearic acid, capric acid, caproic acid, palmitic acid, oleic acid, palmitoleic acid, triacontanoic acid, benzoic acid, methyl benzoic acid, salicylic acid, a rosin acid, abietic acid, phthalic acid, isophthalic acid, terephthalic acid, sebacic acid, adipic acid, azelaic acid, succinic acid, fumaric acid, maleic acid, naphthalene dicarboxylic acid, pamoic acid, trimellitic acid, citric acid, trimesic acid or pyromellitic acid.

24. A process according to claim 22 characterized in that the esterification reaction comprises a reaction of an alcohol with an anhydride of a dicarboxylic acid or a tricarboxylic acid.

25. A process according to claim 22 characterized in that the esterification reaction comprises reaction of a methyl ester, an ethyl ester or a propyl ester of acrylic acid or methacrylic acid with an alcohol.

26. A process according to claim 22 characterized in that the esterification reaction comprises reaction of two esters to produce two different esters by exchange of alkoxy groups.

27. A process according to claim 22 characterized in that the esterification reaction comprises a polyesterification comprising the reaction of terephthalic acid, dimethyl terephthalate, dimethyl naphthalenate or naphthalene dicarboxylic acid with 1,2-ethanediol, 1,4-butanediol, 1,3-propanediol, 1,6 cyclohexane dimethanol, trimethylolpropane or pentaerythritol.

28. A process according to claim 22 characterized in that the catalyst is present in an amount in the range 10 to 1200 parts per million calculated as parts by weight of total metal (Ti, Zr or Al plus Ge, Sb or Sn) with respect to weight of product ester.

29. A process according to claim 22 or 27 characterized in that the esterification reaction is a polyesterification and the catalyst is present in an amount in the range 5 to 550 parts per million calculated as parts by weight total metal (Ti, Zr or Al plus Ge, Sb or Sn) with respect to weight of product polyester.

30. A process according to claim 22, characterized in that the catalyst composition is present in an amount such that the total amount of titanium, zirconium or aluminium present is in the range 5 to 500 parts per million calculated as parts by weight of Ti, Zr or Al with respect to weight of product ester and the total amount of germanium, antimony or tin present is in the range 5 to 700 ppm calculated as Ge, Sb or Sn with respect to product ester.

31. A process according to claim 22, characterized in that the catalyst composition is present in an amount such that the total amount of titanium, zirconium or aluminium present is in the range 3 to 250 parts per million calculated as parts by weight of Ti, Zr or Al with respect to weight of product polyester and the total amount of germanium, antimony or tin present is in the range 3 to 300 ppm calculated as Ge, Sb or Sn with respect to product polyester.

32. A polyester comprising the residues of a reaction between a polybasic acid or ester thereof with a polyhydric alcohol and further containing residues of a catalyst system comprising:
(a) the reaction product of an orthoester or condensed orthoester of at least one metal selected from titanium, zirconium or aluminium, an alcohol containing at least two hydroxyl groups and an organophosphorus compound containing at least one P—OH group, and
(b) at least one compound of germanium, antimony or tin.
